(19) United States
(12) Patent Application Publication
(10) Pub. No.: US 2004/0030120 A1
(43) Pub. Date: Feb. 12, 2004

(54) Thauera strain mz1t exopolysachharides

(76) Inventors: Michael S. Allen, Maryville, TN (US); Gary S. Sayler, Road Blaine, TN (US); Arthur Meyers, Kingsport, TN (US)

Correspondence Address: NEEDLE & ROSENBERG, P.C., SUITE 1000, 999 PEACHTREE STREET, ATLANTA, GA, 30309-3915, US

(21) Appl. No.: 10/240,432

(22) PCT Filed: Apr. 2, 2001
(86) PCT No.: PCT/US01/10657

(51) Int. Cl. C07H 5/04; C07H 5/06; C08B 37/00; C12P 19/00; C07K 1/00; C07K 14/00; C07K 17/00

(52) U.S. Cl. 536/55.1; 536/55.3; 435/72; 530/395

(57) ABSTRACT

The invention provides partially purified EPS that was purified from Thauera sp. Strain MZ1T. This partially purified EPS comprises rhamnose, xylose, galacturonic acid, galactose, glucose, N-acetylfucosamine. The invention further provides methods of producing the partially purified EPS and of utilizing the partially purified EPS in methods of chelating metals. Further provided by this invention is an exopolysaccharide termed “thaueran” that was purified from Thauera sp. Strain MZ1T. This exopolysaccharide has a molecular weight of about 250 kDa and comprises rhamnose, galacturonic acid, N-acetylfucosamine and N-acetylglucosamine. The invention also provides methods of producing the exopolysaccharide and of utilizing the exopolysaccharide in methods of chelating metals.

This application claims priority from provisional patent application Serial No. 60/193,750, filed Mar. 31, 2000, which is hereby incorporated in its entirety by this reference.

### FIELD OF THE INVENTION

The invention relates to the field of polysaccharides. More particularly, the invention relates to a partially purified exopolysaccharide and a purified exopolysaccharide, termed thaueran, produced from a bacterial species. This invention also relates to methods of making and using these exopolysaccharides.

### BACKGROUND

A number of microorganisms produced extracellular polysaccharides, also known as exopolysaccharides or EPS. Complex polysaccharides such as microbial expolysaccharides have a variety of industrial applications. For example, many of these molecules have been used as thickeners in the food and cosmetic industries, adhesives, coating agents, freeze stabilizers, water soluble lubricants, for suspending waste fragments in drilling or cutting operations, and for reducing turbulence of fluid flow in pipelines and other containers. See, e.g., Baird et al., “Industrial Applications Of Some New Microbial Polysaccharides,” Biotechnology, November 1983, pp. 778-83; J. W. Hoyt, Drag Reduction In Polysaccharide Solutions, Trends in Biotechnology, 3, 17-20 (1985); and G. R. Petersen et al. “Rheologically Interesting Polysaccharides From Yeasts”, Appl. Biochem. Biotech., 20/21, 845-67 (1989). In other applications, complex polysaccharides have been used as anti-neoplastic agents, hypertension controlling agents, wound healing agents, food coating agents and enzyme substrates. See, e.g. Sutherland, “Novel and established applications of microbial polysaccharides,” Trenzds in Biotechnology 16, 41-46 (1998); Sutherland, “Industrially Useful Microbial Polysaccharides,” Microbiological Sciences 3, 5-9 (1986) and Vanhooren and Vandamme, “L-Fucose: occurrence, physiological role, chemical, enzymatic and microbial synthesis,” J. Chem Technol. Biotechnol. 74: 479-497 (1999).

Perhaps the most common example of a commercially useful microbial exopolysaccharide is xanthan gum. Xanthan gum is produced by a gram-negative bacterium (Xanthomonas campestris), and is used as a thickener in many different compositions. See, e.g., Jeanes, et al., J. Appl. Polymer Sci., 5,519-526 (1961). Other examples of useful bacterial exopolysaccharides include gellan (a microbial anionic heteropolysaccharide composed of tetrasaccharide units produced by Auromonas elodea ATCC 31461); S-88 (See, Kang and Veeder, U.S. Pat. No. 4,535,153); welan (See, Kang and Veeder, U.S. Pat. No. 4,342,866); NW11 (See, Robison and Stipanovic, U.S. Pat. No. 4,874,044); rhamsan (See, Peik, et al. Al., U.S. Pat. No. 4,401,760); S198 (See, Peik, et al. Al. U.S. Pat. No. 4,529,797); S-657 (See, Peik, et al., Eur. Patent Application 209277A1); and heteropolysaccharide-7 (See, Kang and McNeely, U.S. Pat. No. 4,342,866).

The invention relates to a partially purified bacterial exopolysaccharide and a purified exopolysaccharide with a molecular weight of about 250 kD, termed “thaueran,” that were obtained from Thauera sp. Strain MZIT, an organism previously isolated from the wastewater treatment system of an industrial manufacturing plant.

### SUMMARY OF THE INVENTION

The present invention provides a partially purified exopolysaccharide produced from Thauera strain MZ1T, that comprises rhamnose, xylose, galacturonic acid, galactose, glucose, N-acetylfucosamine and N-acetylglucosamine.

The present invention also provides an isolated exopolysaccharide, termed thaueran, produced from Thauera strain MZ1T. This exopolysaccharide has a molecular weight of about 250 kDa and comprises rhamnose, galacturonic acid, Nacetylfucosamine and N-acetyl glucosamine.

Also provided by the present invention is a method of producing partially purified exopolysaccharide produced from Thauera strain MZ1T comprising the steps of: a) culturing the Thauera MZ1T in media; and b) purifying the exopolysaccharide produced from the Thauera MZ1T culture of step a).

This invention further provides a method of producing thaueran EPS produced from Thauera strain MZ1T comprising the steps of: a) culturing the Thauera MZ1T in media; and b) purifying the thaueran EPS produced from the Thauera MZ1T culture of step a).

Further provided by the present invention is a method of removing metal from a sample comprising: a) contacting a metal containing sample with a partially purified EPS produced from Thauera strain MZ1T; b) allowing a complex to form between the metal in the metal containing sample and the partially purified EPS; and c) removing the complex of step b) from the sample.

Also provided by the present invention is a method of removing metal from a sample comprising: a) contacting a metal containing sample with thaueran EPS produced from Thauera strain MZ1T; b) allowing a complex to form between the metal in the metal containing sample and the thaueran EPS; and c) removing the complex of step b) from the sample.

### BRIEF DESCRIPTION OF THE DRAWINGS

FIG. 1 is a chart showing results obtained in a one dimensional NMR analysis of partially purified exopolysaccharide.

FIG. 2 is a chart showing results obtained in a two dimensional NMR analysis (gCOSY) of partially purified exopolysaccharide.

FIG. 3 is a chart showing results obtained in a two dimensional NMR analysis (NOESY) of partially purified exopolysaccharide.

FIG. 4 is a chart showing results obtained in an IR analysis of partially purified exopolysaccharide.

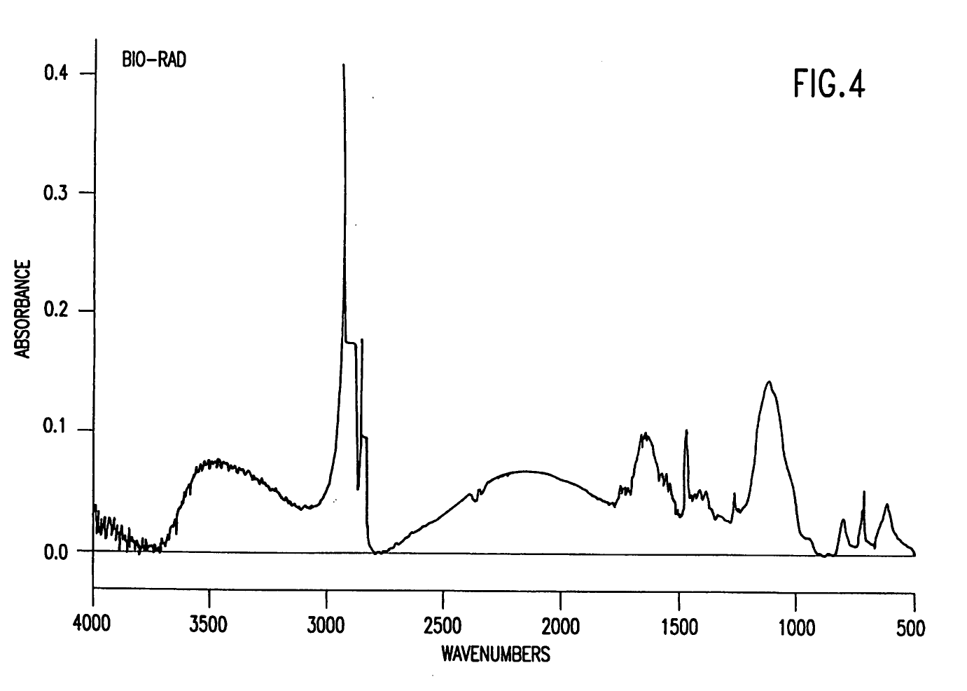

FIG. 5 shows an IR transmittance spectrum for 250 kD thaueran exopolysaccharide.

FIG. 6 is a chart showing the results obtained in a one dimensional ¹H-NMR analysis of 250 kD thaueran exopolysaccharide.

FIG. 7 is a chart showing the results obtained in a two dimensional NMR analysis (gCOSY) of 250 kD thaueran exopolysaccharide.

FIG. 8 is a chart showing the results obtained in a two dimensional NMR analysis (TOCSY) of 250 kD thaueran exopolysaccharide.

FIG. 9 is a chart showing the results obtained in a two dimensional NMR analysis (NOESY) of 250 kD thaueran exopolysaccharide.

FIG. 10 is a chart showing the results obtained in a two dimensional ¹³C-¹H NMR analysis (HSQC) of 250 kD thaueran exopolysaccharide.

### DETAILED DESCRIPTION OF THE INVENTION

The present invention may be understood more readily by reference to the following detailed description of the preferred embodiments of the invention and the Examples included herein.

Before the present methods are disclosed and described, it is to be understood that this invention is not limited to specific polysaccharides or specific methods. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise.

The present invention provides a partially purified exopolysaccharide (EPS) produced from Thauera strain MZ1T. Monosaccharide constituents of the partially purified exopolysaccharide include glucose, galacturonic acid, and N-acetylfucosamine in a ratio of approximately 4:2:1. Additionally, the partially purified EPS contains the monosaccharides rhamnose, xylose, and galactose in lower quantities. Methylation analysis showed that the partially purified EPS is highly branched having 1-3, 14, and 1-6 glycosyl linkages.

MZ1T produces a partially purified EPS when grown on simple organic acids and related compounds commonly associated with industrial wastewaters. After purification, the partially purified EPS appears as a white fibrous material with a cotton-like consistency. The partially purified EPS is readily soluble in water, but insoluble in most organic solvents and solubility is reduced above pH 7.

The present invention also provides a method of producing the partially purified EPS from Thauera MZ1T comprising the steps of: a) culturing the Thauera MZ1T in media; and b) purifying the partially purified EPS produced from the Thauera MZ1T culture of step a). Microorganism culturing techniques are well known in the art and the Examples included herein provide sufficient guidance for one of skill in the art to make the appropriate media, culture MZ1T and recover the partially purified EPS of the present invention. For example, MZ1T can be grown in most non-selective, complex media including YEPG (Yeast extract, peptone, and glucose media). Stoke's medium can also be utilized.

In the method described above, the partially purified EPS can be purified by: a) centrifuging the Thauera MZ1T culture; b) decanting the supernatant; c) concentrating the supernatant; d) dialyzing the supernatant against water using molecular weight cutoff dialysis; e) collecting the contents of the dialysis tubes.

Further provided by the present invention is a method of chelating a metal in a sample, comprising the steps of: a) contacting a metal containing sample with a partially purified EPS produced from Thauera strain MZ1T and b) allowing a complex to form between the metal in the metal containing sample and the partially purified EPS, therefore chelating a metal in the sample.

The present invention also provides a method of removing metal from a sample comprising: a) contacting a metal containing sample with a partially purified EPS produced from Thauera strain MZ1T; b) allowing a complex to form between the metal in the metal containing sample and the partially purified EPS; and c) removing the complex of step b) from the sample.

The metals that can be chelated and/or removed by these methods include, but are not limited to uranium, calcium, cadmium, copper and other positively charged metal cations.

Since the partially purified EPS is capable of chelating metals, it is specifically envisioned to be useful for removing metal ions from aqueous and non-aqueous solutions resulting from industrial processes or a waste stream. The partially purified EPS/wastewater is mixed to allow the dissolved partially purified EPS to bind metals present in the wastewater. A precipitating agent such as a calcium salt (CaCl₂ precipitates the partially purified EPS dissolved in aqueous solutions) or a base is then added to precipitate the partially purified EPS out of solution. This precipitate is separated from the water by, for example, allowing the precipitate to sediment to the bottom of the tank and removing the purified wastewater from the top of the tank. The precipitate can also be separated from the water by filtration. The metal-containing partially purified EPS is then disposed. In some applications, the partially purified EPS can be treated with an acid to free the bound metals, or the metals can be precipitated by reacting them with an anion (sulfate for example, depending on the metal of interest) and neutralizing pH to form insoluble metal salts. Free sugars resulting from these processes can be washed into the wastestream. It is also envisioned that heavy metals bound by the partially purified EPS could be exchanged with another metal so that the partially purified EPS could be safely reused. Additionally, it is envisioned that because partially purified EPS is insoluble in organics (solvents, alcohols, etc.), a woven filter could be made to allow flow through metal extraction from solvents and solutions similar to the filter utilized to separate a metal-partially purified EPS precipitate from water. Given the polymeric nature of the partially purified exopolysaccharide, it is expected that it can be weaved and manipulated to generate cellulose based filters.

The partially purified EPS of this invention is similar to other bacterial polysaccharides that have been used in hydrating agents, stabilizing emulsions, gel formulation, viscosity control, foam stabilization, film formation, suspending agents, acoustical membranes, and structural fibers. The partially purified EPS is therefore expected to be useful in one or more of these applications as well. Given the similarity of partially purified EPS to other polysaccharides with known uses, it is specifically envisioned that partially purified EPS could be used commercially for food and cosmetic uses, oil extraction, as components of firefighting fluids, as wound dressings in eye and joint surgery, and as anti-tumor agents. See e.g. Sutherland, “Novel and established applications of microbial polysaccharides,” Trends in Biotechnology 16, 41-46 (1998); Sutherland, “Industrially Useful Microbial Polysaccharides,” Microbiological Sciences 3, 5-9 (1986) and Vanhooren and Vandamme, “L-Fucose: occurrence, physiological role, chemical, enzymatic and microbial synthesis,” J. Chem Technol. Biotechnol. 74: 479497 (1999). This partially purified EPS is also useful as an agent for promoting flocculation in waste water treatment.

In addition, because of its fibrous nature, partially purified EPS could be woven into a product that would dissolve in the presence of water. The solubility could be controlled (at least temporarily) by controlling the calcium concentration in the final product. Partially purified EPS might thus be useful for making cigarette filters that degrade in the environment to reduce pollution, or as line filters for solvents. Partially purified EPS could also be used for extraction of various substances from solvents and transferred to an aqueous solution to dissolve the polymer and free up a material of interest.

The present invention also provides an isolated exopolysaccharide produced from Thauera strain MZ1T, termed thaueran. More specifically, the invention provides an isolated exopolysaccharide produced from MZ1T, wherein the exopolysaccharide has a molecular weight of about 250 kDa and comprises rhamnose, galacturonic acid, N-acetylfucosamine and N-acetyl glucosamine.

Monosaccharide constituents include rhamnose (Rha), galacturonic acid (GalA), N-acetylfucosamine (2-acetamido-2,6-dideoxy-galactose, FucNac) and N-acetyl glucosamine.

MZ1T produces thaueran EPS when grown on simple organic acids and related compounds commonly associated with industrial wastewaters. After purification, the thaueran EPS appears as a white fibrous material with a cotton-like consistency. The thaueran EPS is readily soluble in water, but insoluble in most organic solvents and solubility is reduced above pH 7.

The present invention also provides a method of producing the thaueran EPS from Thauera MZ1T comprising the steps of: a) culturing the Thauera MZ1T in media; and b) purifying the thaueran EPS produced from the Thauera MZ1T culture of step a).

Microorganism culturing techniques are well known in the art and the Examples included herein provide sufficient guidance for one of skill in the art to make the appropriate media, culture MZ1T and recover the thaueran EPS of the present invention.

In the method described above, the thaueran EPS can be purified by: a) centrifuging the Thauera MZ1T culture; b) decanting the supernatant; c) concentrating the supernatant; d) purifying the supernatant by size exclusion chromatography; e) collecting a thaueran EPS containing fraction produced by step (d), thus purifying the thaueran EPS.

Further provided by the present invention is a method of chelating a metal in a sample, comprising the steps of: a) contacting a metal containing sample with thaueran EPS produced from Thauera strain MZ1T and b) allowing a complex to form between the metal in the metal containing sample and the thaueran EPS, therefore chelating a metal in the sample.

The present invention also provides a method of removing metal from a sample comprising: a) contacting a metal containing sample with thaueran EPS produced from Thauera strain MZ1T; b) allowing a complex to form between the metal in the metal containing sample and thauean EPS; and c) removing the complex of step b) from the sample.

The metals that can be chelated and/or removed by these methods include, but are not limited to uranium and a number of positively charged metal cations. See, e.g. Carol et al. Environ. Sci. Tech 33:3622-3626 (1999). Also, the presence of galacturonic acid allows thaueran EPS to bind positively charged substances and the presence of N-acetylfucosamine allows thaueran EPS to bind negatively charged substances. Therefore, thaueran EPS can be used to bind both positively and negatively charged substances.

Similar to the partially purified EPS, thaueran EPS is specifically envisioned to be useful for removing metal ions from aqueous and non-aqueous solutions resulting from industrial processes or a waste stream. The thaueran EPS/wastewater is mixed to allow the dissolved thaueran EPS to bind metals present in the wastewater. A precipitating agent such as a calcium salt (CaCl₂ precipitates the thaueran EPS dissolved in aqueous solutions) or a base is then added to precipitate the thaueran EPS out of solution. This precipitate is separated from the water by, for example, allowing the precipitate to sediment to the bottom of the tank and removing the purified wastewater from the top of the tank. The precipitate can also be separated from the water by filtration. The metal-containing thauean EPS is then disposed. In some applications, the thaueran EPS can be treated with an acid to free the bound metals, or the metals can be precipitated by reacting them with an anion (sulfate for example, depending on the metal of interest) and neutralizing pH to form insoluble metal salts. Free sugars resulting from these processes can be washed into the wastestream. It is also envisioned that heavy metals bound by the thaueran EPS could be exchanged with another metal so that the thaueran EPS could be safely reused. Additionally, it is envisioned that because thaueran EPS is insoluble in organics (solvents, alcohols, etc.), a woven filter could be made to allow flow through metal extraction from solvents and solutions similar to the filter utilized to separate a metal-thaueran EPS precipitate from water.

The thaueran EPS of this invention is similar to other bacterial polysaccharides that have been used in hydrating agents, stabilizing emulsions, gel formulation, viscosity control, foam stabilization, film formation, suspending agents, acoustical membranes, and structural fibers. The thaueran EPS is therefore expected to be useful in one or more of these applications as well. Given the similarity of thaueran EPS to other polysaccharides with known uses, it is specifically envisioned that thaueran EPS could be used commercially for food and cosmetic uses, oil extraction, as components of firefighting fluids, as wound dressings in eye and joint surgery, and as anti-tumor agents. See e.g. Sutherland, “Novel and established applications of microbial polysaccharides,” Trends in Biotechnology 16, 41-46 (1998); Sutherland, “Industrially Useful Microbial Polysaccharides,” Microbiological Sciences 3, 5-9 (1986) and Vanhooren and Vandamme, “L-Fucose: occurrence, physiological role, chemical, enzymatic and microbial synthesis,” J. Chem Technol. Biotechnol. 74: 479-497 (1999). This thaueran EPS is also useful as an agent for promoting flocculation in waste water treatment.

In addition, because of its fibrous nature, thaueran EPS could be woven into a product that would dissolve in the presence of water. The solubility could be controlled (at least temporarily) by controlling the calcium concentration in the final product. Thaueran EPS might thus be useful for making cigarette filters that degrade in the environment to reduce pollution, or as line filters for solvents. Thaueran EPS could also be used for extraction of various substances from solvents or in a water bath to free up a material of interest.

The presence of N-acetylfucosamine (2-acetamido-2, 6-dideoxy-galactose, FucNac) in thaueran makes thaueran EPS a valuable source of fucose. Since fucose is costly and difficult to synthesize, thaueran EPS represents a source material from which fucose can be obtained for numerous anti-neoplastic and hypoallergenic applications. Fucose containing oligosaccharides can be used to prevent tumor cell colonization of the lung, to control formation of white blood cells (anti-inflammatory effect) and to treat rheumatoid arthritis (Vanhooren and Vandamme “L-Fucose: Occurrence, physiological role, chemical, enzymatic and microbial synthesis” J. Chem Technol. Biotechnol. 74: 479-497 (1999). Fucose can also be used in the synthesis of antigens for antibody production and in cosmetics as a skin moisturizing agent. Since fucose production via chemical synthesis is laborious and suffers from low yield, fucose can be obtained from thaueran by chemical or enzymatic hydrolysis.

### EXAMPLES

MZ1T

MZ1T is a floc-forming bacterium previously isolated from an industrial wastewater treatment plant as described in Lajoie et al., Water Environment Research, 72:56, 2000. MZIT has been deposited with the American Type Culture Collection as Accession number ATCC No. 202120.

Materials

Thauera defined medium (TDM) which was used to culture growth was prepared as described below.

A trace elements solution was prepared by mixing together the following:

| | | |
|---|---|---|
| FeSO₄.7H₂O | 2100 | mg |
| Na₂EDTA | 5200 | mg |
| H₃BO₃ | 30 | mg |
| MnCl₂.4H₂O | 100 | mg |
| CoCl₂.6H₂O | 190 | mg |
| NiCl₂.6H₂O | 24 | mg |
| CuSO₄.5H₂O | 25 | mg |
| ZnSO₄.7H₂O | 144 | mg |
| NaMoO₄.2H₂O | 36 | mg |
| Add H₂O to bring to | 1000 | ml |

The pH of the resulting solution was adjusted to between 6.0-6.5 with NaOH A W (tungsten)-solution was prepared by mixing together the following:

| | | |
|---|---|---|
| NaOH | 200 | mg |
| Na₂WO₄2H₂O | 8 | mg |
| Add H₂0 to bring to | 1000 | ml. |

A Vitamin mixture was prepared by mixing together the following:

| | | |
|---|---|---|
| Sodium phosphate-buffer, | 200 | ml |
| 20 mM, pH = 7.1 | | |
| p-aminobenzoic acid | 8 | mg |
| D(+)Biotin | 2 | mg |
| Nicotinic acid | 20 | mg |
| Ca-D(+)Pantothenate | 10 | mg |
| Pyridoxin-Hydrochloride | 30 | mg. |

A Thiamin solution was prepared by mixing together the following:

| | | |
|---|---|---|
| Sodium phosphate-buffer, | 200 | ml |
| 25 mM, pH = 7.1 | | |
| Thiamine-Chloride-Dihydrochloride | 200 | mg. |

A Vitamin B₁₂-solution was prepared by mixing together the following:

| | | |
|---|---|---|
| add H₂O | 100 | ml |
| Cyanocobalamine | 5 | mg |

Nitrocellulose filters with a pore size of 0.2 μm were used for the sterile filtration of all vitamin solutions.

A Sodium bicarbonate solution (1 M) was prepared by mixing together the following:

| | | |
|---|---|---|
| NaHCO₃ | 84 | g |
| Add H₂O to bring to | 1000 | ml |

One liter of solution was autoclaved in a sealed screw top container.

Freshwater medium (for aerobic growth conditions) was prepared by mixing together the following:

| | | |
|---|---|---|
| NH₄Cl | 0.3 | g |
| KH₂PO₄ (monobasic) | 0.5 | g |
| MgSO₄7H₂O | 0.5 | g |
| CaCl₂2H₂O | 0.1 | g |
| Sodium succinate | 5 | g |
| Add H₂O to bring to | 1000 | ml |

(0.85 g NaNO₃ can be added for anaerobic growth
conditions)

This freshwater medium was autoclaved in a 1.5 L bottle, which was then sealed with a screw cap.

After the freshwater medium in 1.5 L bottle medium cooled, TDM was made by adding the following sterile solutions to 1 L of the freshwater medium:

| | | |
|---|---|---|
| Trace element solution | 1 | ml |
| W-solution | 1 | ml |
| Sodium bicarbonate-solution (1 M) | 30 | ml |
| Vitamin mixture | 1 | ml |
| Thiamin solution | 1 | ml |
| B₁₂ solution | 1 | ml |

Purification of Partially Purified EPS

Partially purified EPS was purified as described below. 1 L of TDM containing 100 mg/l rifampicin was inoculated with 200 ul of frozen MZ1T rifampicin resistant stock culture (a rif-resistant mutant of the wild type MZ1T was generated by repeated plating and screening for growth on rif plates; wild type MZ1T has also been used to prepare purified thaueran EPS in the same manner but without adding rifampicin to the medium). The inoculated medium was incubated at 30° C. in a shaking incubator (160 rpm) for 7 days. After this time, the culture was centrifuged at 10,000×g for 15 minutes to pellet the bacteria. The supernatant was decanted, collected, and then concentrated to approximately 200 ml by rotary evaporation at 45° C. under reduced pressure. The concentrate was then dialyzed against 4 L of water using 3500 Da molecular weight cutoff (MWCO) dialysis tubes. The water was changed 7 times over 3 days (about every 6-10 hours). The contents of the dialysis tubes was then transferred to fresh falcon tubes, frozen at −80 degrees C. for >1 h, and then lyophilized until dry (˜3 days) to yield the partially purified EPS.

Characterization of the Partially Purified EPS

A sample of partially purified EPS was purified as described above and analyzed by conventional chemical analyses. In solubility experiments, partially purified EPS proved readily soluble in water at pHs below about 7. Partially purified EPS was less soluble at higher pHs. The solubility of partially purified EPS in other solvents was investigated. Partially purified EPS proved insoluble in DMSO, acetone, ethanol, methanol, hexane, and ethyl acetate. It was also noted that the addition of CaCl₂ (0.25-0.3 M) caused partially purified EPS to precipitate out of aqueous solutions. In melting point analyses, partially purified EPS decomposed at temperatures above 205° C. In viscosity analyses, it was noted that aqueous solutions became more viscous as higher concentrations of partially purified EPS were added.

Partially purified EPS was also analyzed for carbohydrate content. A sample of partially purified EPS was hydrolyzed using freshly prepared 1 M methanolic-HC1 for 16h at 80° C. The released sugars were derivatized with Tri-Sil and the samples were analyzed by Gas Chromatography (GC) using a Supelco column. Myo-inositol (20 ug) was also added as an internal standard. Results are shown in Table 1 below.

TABLE 1

| Carbohydrate Residues | Mole |
|---|---|
| Rhamnose | 7.9 |
| Xylose | 1.3 |
| Galacturonic acid | 25.5 |
| Galactose | 3.4 |
| Glucose | 49.5 |
| N-acetylfucosamine | 12.3 |

The percentage of soluble carbohydrate in the same was calculated to be 24.9%. The sample contained about 50% glucose and GalA as its major components. The sample also contained an unusual sugar residue N-acetylfucosamine. Also contained in this partialy purified EPS sample was N-acetylglucosamine.

Glcosyl linkage was assessed by conventional techniques using methylation analysis-GC mass spectrometry. Briefly, a partially purified EPS sample was dissolved in DMSO three days prior to the methylation. It was stirred and sonicated with 50 μl of glycerol to facilitate dissolution in the DMSO. The sample was methylated by the Hakomori procedure and then reduced with superdeuteride for 3h at room temperature. The methylated sample was hydrolyzed with 2M TFA at 120° C. for 2 h, and then reduced using sodium borodeuteride. The reduced sample was acetylated by pyridine and acetic anhydride prior to GC-MS analysis. Myoinositol was also added to the sample prior to the reduction step.

Results of this analysis are shown below in Table 2:

TABLE 2

| Carbohydrate Residues | % present |
|---|---|
| Terminal glucose | 2.8 |
| 4-xylose | 10.6 |
| 3-glucose | 18.0 |
| 3-galacturonic acid | 17.7 |
| 6-glucose | 47.0 |
| 4-galactose | 3.9 |

Under the conditions of this experiment, rhamnose was not detected in the sample. This could be due to the high volatility of rhamnose residues that could either be t-rhamnose or 2-rhamnose. FucNac would also not be detected under these conditions. The results suggested that the partially purified EPS is highly branched having 1-3,14, and 1-6 glycosyl linkages.

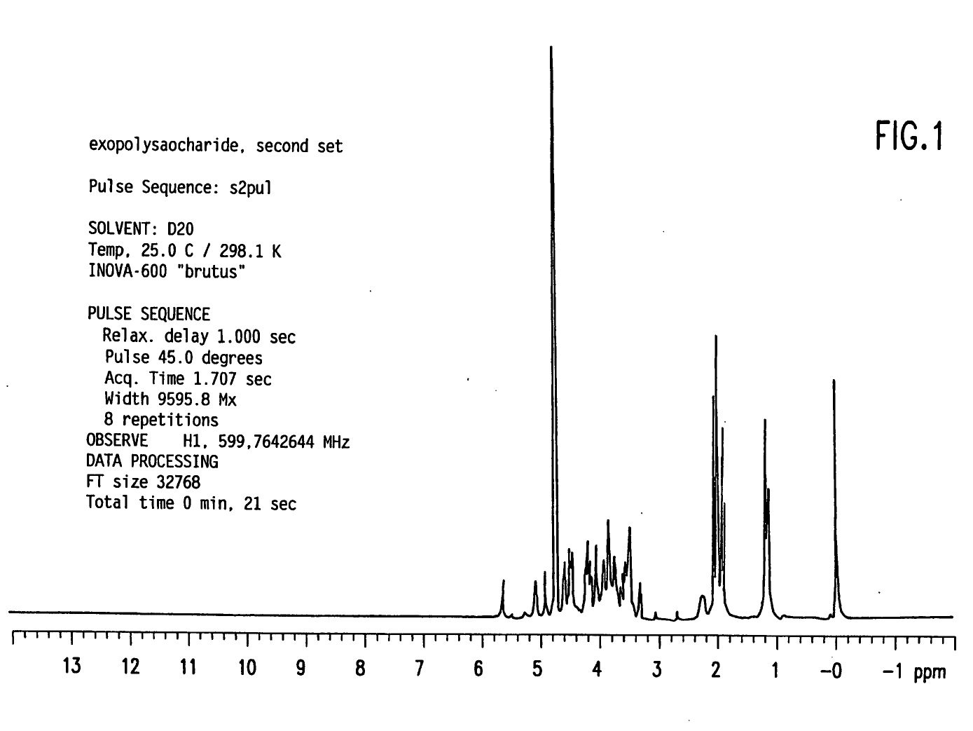

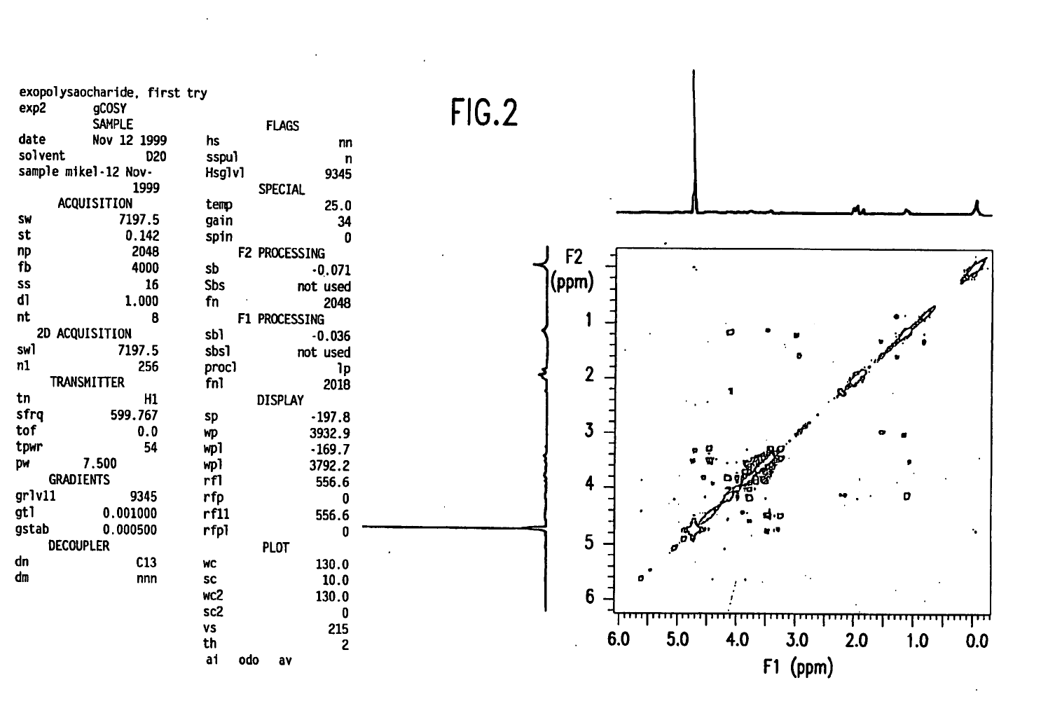

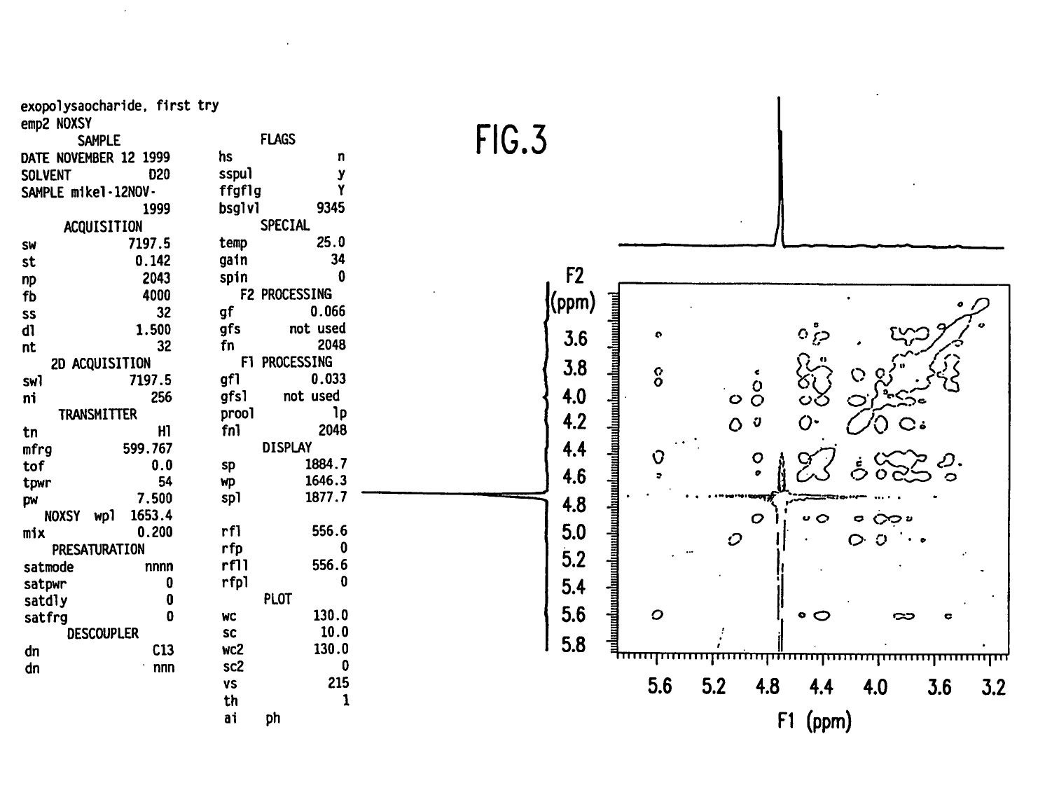

The structure of partially purified EPS was also characterized using NMR spectroscopy and IR spectroscopy. FIG. 1 shows results obtained in a one dimensional NMR analysis. FIG. 2 shows results obtained in a two dimensional NMR analysis (gCOSY). FIG. 3 shows results obtained in a two dimensional NMR analysis (NOESY). And FG. 4 shows results obtained in an IR analysis.

Metal Chelation

Partially purified EPS was shown to chelate uranium (U) under a variety of conditions. In one experiment, uranium binding to partially purified EPS in water was examined. A U stock solution was prepared by dissolving 0.1 g UO₂(NO₃)₂ in 50 ml millipore water (2000 ppm). In a separate container, 10 mg of purified partially purified EPS was dissolved in 1 ml of water. 1 ml of the U stock solution was added to the 1 ml partially purified EPS solution to form a mixture. The mixture was vortexed for 30 seconds, allowed to sit 10 minutes, and then added to a centrifugal filtration column (Millipore Biomax 30-50 Kda filtration column). In a parallel control run, a sample containing only 1 ml water and 1 ml of the U stock was treated in the same manner as the partially purified EPS containing mixture. The filter columns were then centrifuged at 3400 rpm (in a standard centrifuge) for 1 hr. 1.5 ml of the filtered liquid in each sample was transferred into 10 ml of ReadySafe Scintillation cocktail, and the samples were analyzed for radioactivity using a scintilation counter. Table 3 shows the results from 4 runs in cpm.

TABLE 3

| | Partially Purified |
|---|---|
| Control | EPS Present |
| 463.5 | 314 |
| 464.5 | 331 |
| 464.5 | 302 |
| 448.5 | 337.5 |

The results indicated that an average of 30.2% of the originally present uranium was bound by the polysaccharide.

In related experiments, the ability of partially purified EPS to chelate uranium dissolved in methanol was examined. A U stock solution was prepared by dissolving 0.1 g in 50 ml methanol. In each of several test tubes, 8 mg of partially purified EPS was added to 2 ml of the U stock solution to form a mixture (partially purified EPS does not dissolve in methanol). Control samples not containing partially purified EPS were run in parallel. These mixtures were placed in a 30 degree shaking incubator (160 rpm) for 2 hours, after which the tubes were centrifuged at 3000 rpm for 5 minutes to pellet the partially purified EPS. The supernatant was decanted and collected. 1.5 ml of the supernatant from each tube was added to 10 ml ReadySafe Scintillation Cocktail, and the same were analyzed for radioactivity using a scintillation counter. Table 4 shows the results of 6 runs in cpm (10 minutes of counting).

TABLE 4

| C ntrol | Thaueran Present |
|---|---|
| 22471 | 5207 |
| 22328 | 5207 |
| 22852 | 5411 |
| 22999 | 5570 |
| 21095 | 5532 |
| 20915 | 5476 |
| avg. = 22110 | avg. = 5400.5 |

The results indicated that an average of 75.6% of the originally present uranium was removed by the polysaccharide.

Other assays were performed to assess uranium-binding by purified thaueran exopolsaccharide in the presence of equimolar (to U) amounts of EDTA (commonly found in metal-containing waste streams). These experiments were performed using the centrifugation column protocol described above. The results indicated that an average of about 40% of the originally present uranium was bound by the polysaccharide.

Purification of thaueran EPS One liter of seven-day-old MZ1T culture grown in TDM was centrifuged at 8000×g to pellet cells. The resultant cell-free supernatant was decanted and subsequently concentrated to one tenth of its original volume using a Millipore Pellicon 2 Mini Holder filtration apparatus equipped with a 100,000 Da MWCO filter. The retentate containing the thaueran EPS was then brought back to its original volume with deionized water and the process was repeated for a total of three times. The desalted EPS solution was then concentrated to approximately 100 ml, frozen at −70° C., and lyophilized to yield the crude polymer. Crude thaueran EPS was further purified by size exclusion chromatography using a 1 m×2.5 cm column packed with Bio-Gel P-30 fine polyacrylamide gel (Bio-Rad) as per the manufacturer's instructions. The buffer used for all purification was 0.25 M ammonium formate. The buffer was degassed under vacuum and maintained in a reservoir sparged with nitrogen. Approximately 10 ml fractions were collected every hour using an ISCO Retriever m fraction collector. Peaks were detected with an in-line mounted ISCO UA-5 Absorbance/Fluorescence Detector with a 254 nm wavelength filter. Individual fractions were analyzed by spectrophotometry at 214 nm and samples absorbing at this wavelength were frozen and lyophilized to remove ammonium formate. All fraction-containing vials were washed with a total of 10 ml deionized water and the collected wash water was refrozen and lyophilized to concentrate the thaueran EPS.

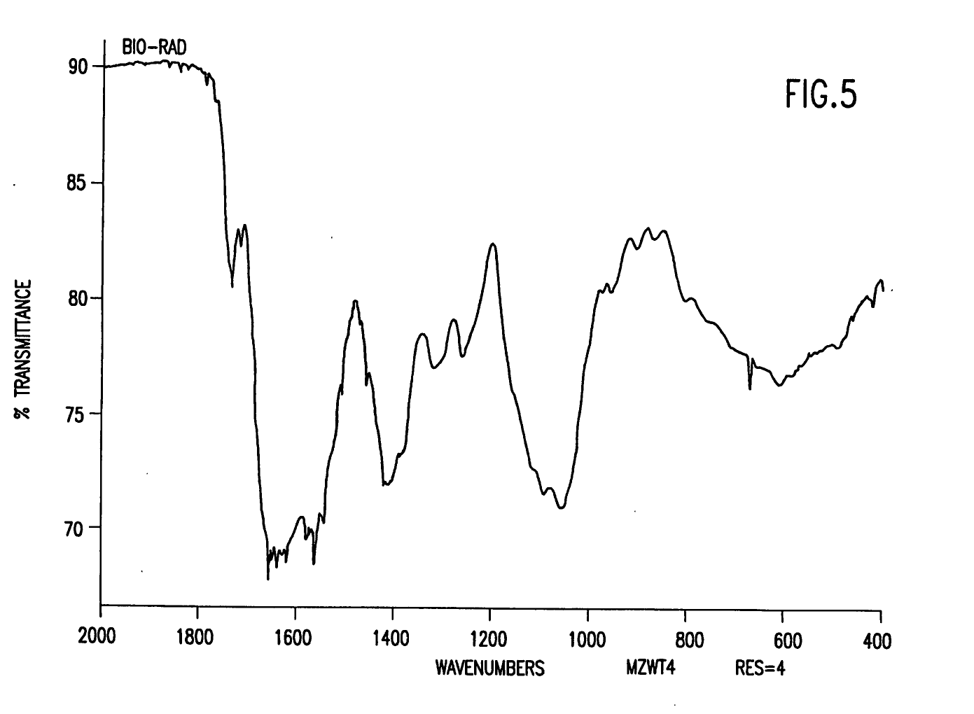

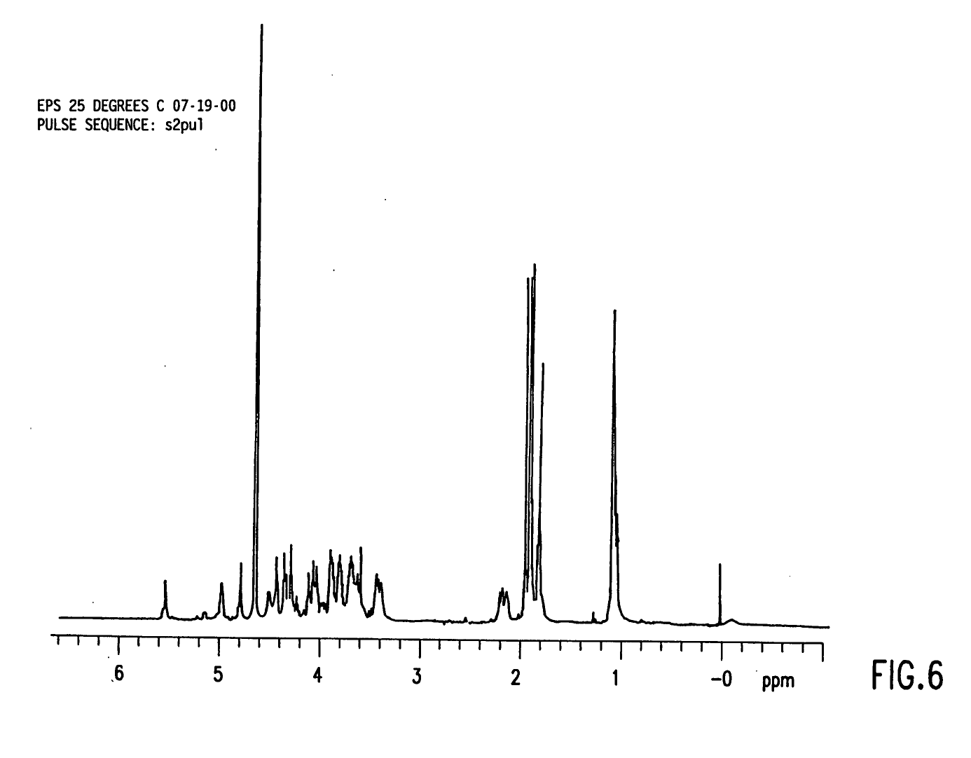

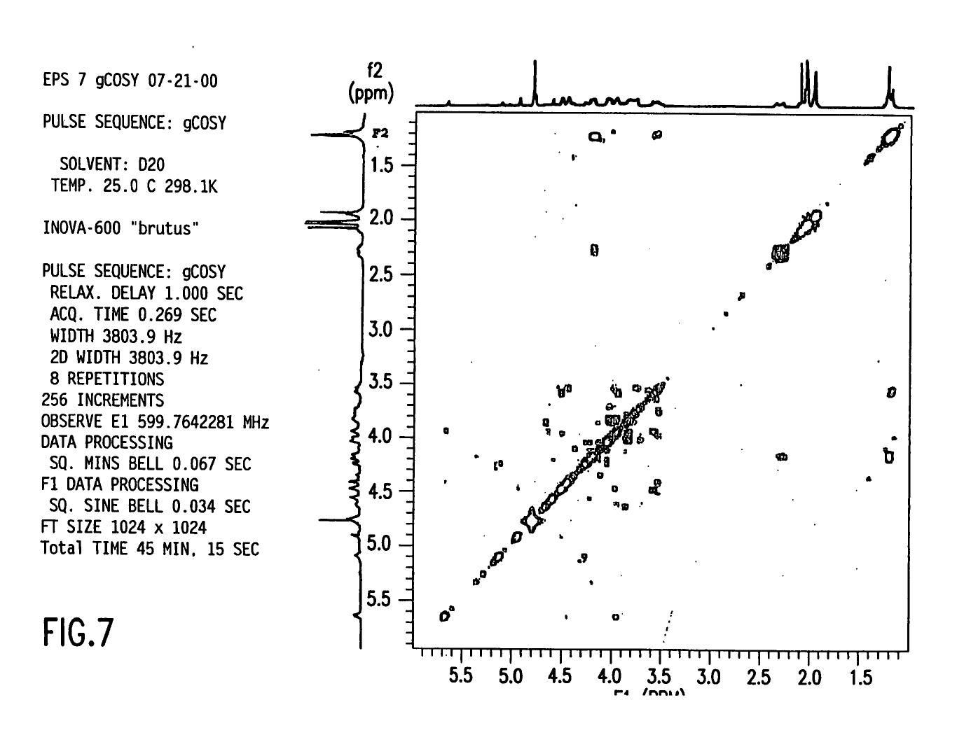

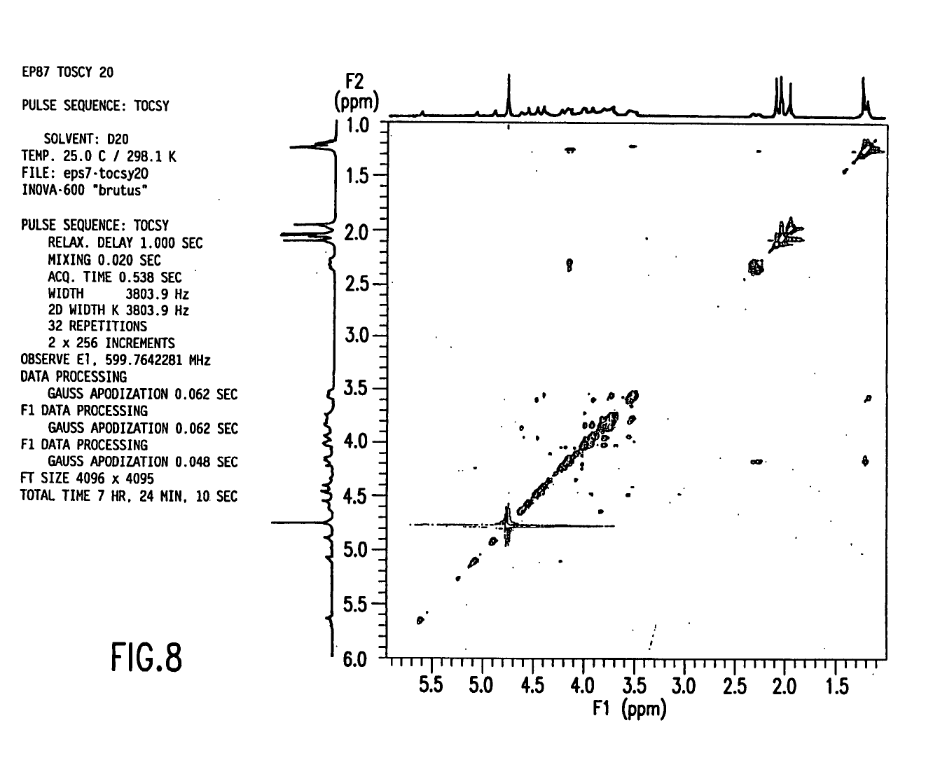

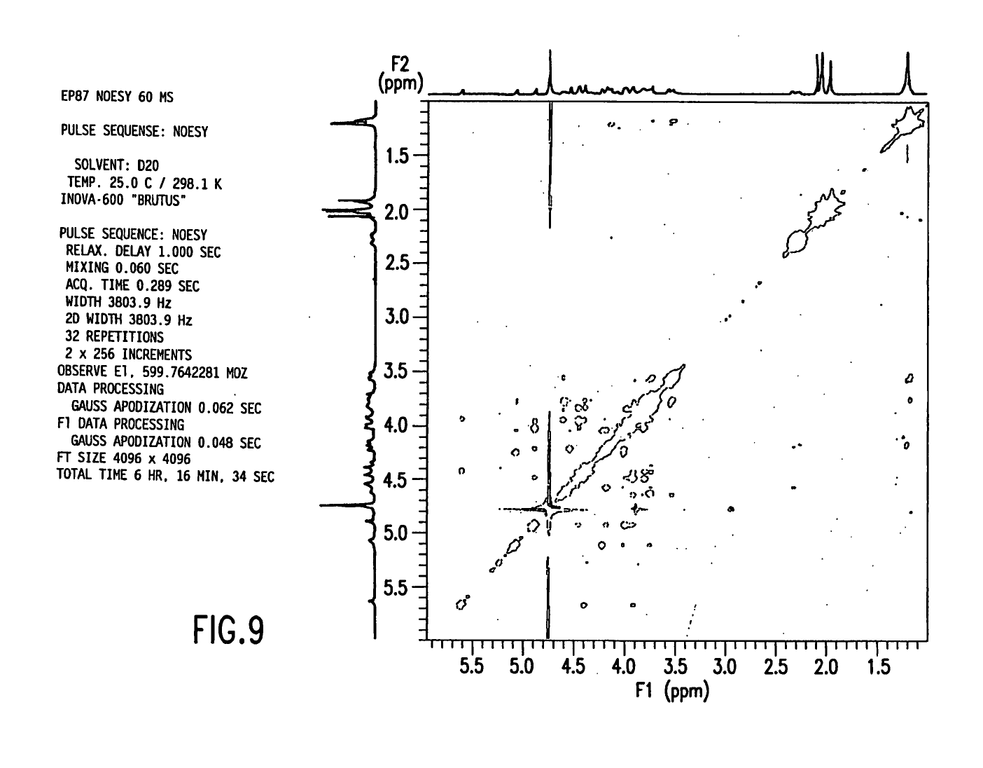

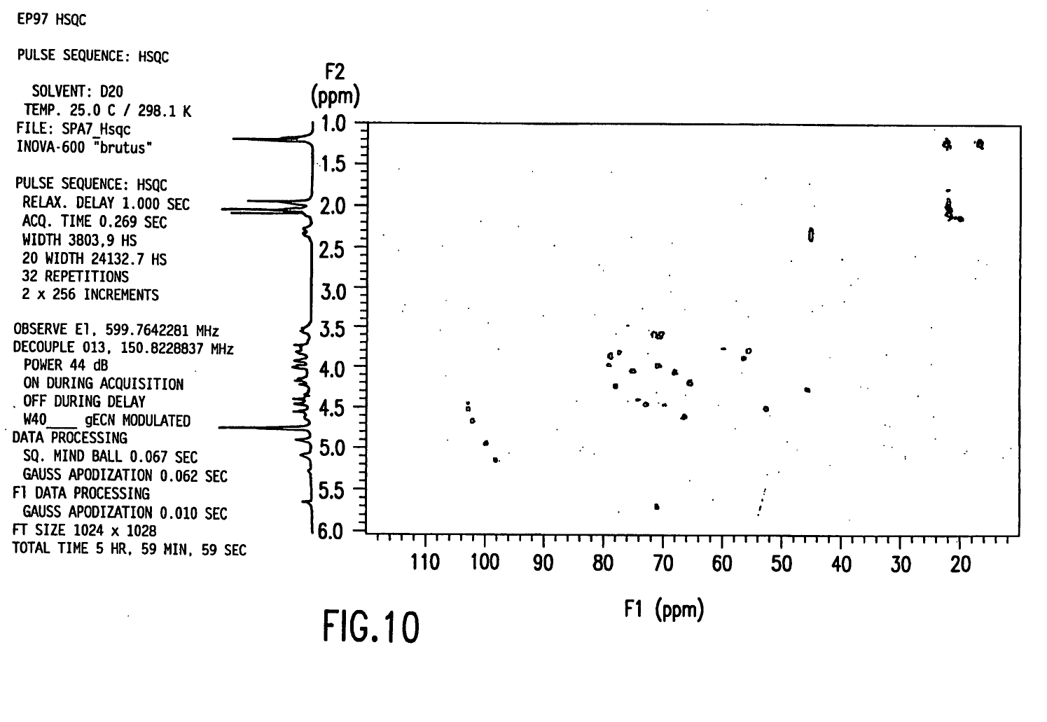

The structure of thaueran EPS was also characterized using NMR spectroscopy and IR spectroscopy. FIG. 5 shows an IR transmittance spectrum for 250 kD thaueran exopolysaccharide. Results obtained in a one dimensional ¹H-NMR analysis of 250 kD thaueran exopolysaccharide are shown in FIG. 6. Two dimensional NMR analysis (gCOSY) of 250 kD thaueran exopolysaccharide is shown in FIG. 7. FIG. 8 is a chart showing the results obtained in a two dimensional NMR analysis (TOCSY) of 250 kD thaueran exopolysaccharide. FIG. 9 is a chart showing the results obtained in a two dimensional NMR analysis (NOESY) of 250 kD thaueran exopolysaccharide. FIG. 10 is a chart showing the results obtained in a two dimensional ¹³C-¹H NMR analysis (HSQC) of 250 kD thaueran exopolysaccharide.

Carbohydrate content of thaueran EPS is analyzed as described above for partially purified EPS. A sample of thaueran EPS is hydrolyzed using freshly prepared 1 M methanolic-HC1 for 16h at 80° C. The released sugars are derivatized with Tri-Sil and the samples are analyzed by Gas Chromatography (GC) using a Supelco column. Myo-inositol (20 ug) is also added as an internal standard. Glycosyl linkage for thaueran EPS is assessed, as described above for partially purified EPS, by conventional techniques using methylation analysis-GC mass spectrometry. Briefly, a thaueran EPS sample is dissolved in DMSO three days prior to the methylation. It is stirred and sonicated with 50 μl of glycerol to facilitate dissolution in the DMSO. The sample is methylated by the Hakomori procedure and then reduced with super deuteride for 3 h at room temperature. The methylated sample is hydrolyzed with 2M TFA at 120° C. for 2 h, and then reduced using sodium borodeuteride. The reduced sample was acetylated by pyridine and acetic anhydride prior to GC-MS analysis. Myoinositol is also added to the sample prior to the reduction step.

Metal chelation experiments for thaueran EPS are performed as described above for partially purified EPS. Due to the presence of metal chelating sugars on thaueran EPS, this exopolysaccharide is expected to have metal chelating properties, such as the ability to chelate uranium.

Although the present process has been described with reference to specific details of certain embodiments thereof, it is not intended that such details should be regarded as limitations upon the scope of the invention except as and to the extent that they are included in the accompanying claims.

Throughout this application, various publications are referenced. The disclosures of these publications in their entireties, as well as the references cited in these publications, are hereby incorporated by reference into this application in order to more fully describe the state of the art to which this invention pertains.

What is claimed is:

1. An isolated exopolysaccharide produced from Thauera strain MZ1T.

2. The exopolysaccharide of claim 1 wherein the exopolysaccharide is a partially purified EPS and comprises rhamnose, xylose, galacturonic acid, galactose, glucose, N-acetylfucosamine and N-acetylglucosamine.

3. The exopolysaccharide of claim 1, wherein the exopolysaccharide has a molecular weight of about 250 kDa and comprises rhamnose, galacturonic acid, N-acetylfucosamine and N-acetylglucosamine.

4. A method of producing the exopolysaccharide of claim 2 comprising the steps of:
a) culturing the Thauera MZ1T in medium; and
b) purifying the partially purified EPS produced from the Thauera MZ1T culture of step a).

5. The method of claim 4, wherein the partially purified EPS is purified by:
a) centrifuging the Thauera MZ1T culture;
b) decanting the supernatant;
c) concentrating the supernatant;
d) dialyzing the supernatant against water using molecular weight cutoff dialysis tubes.
e) collecting the contents of the dialysis tubes.

6. A method of removing a metal from a sample comprising:
a) contacting a metal containing sample with the partially purified EPS of claim 2;
b) allowing a complex to form between the metal in the metal containing sample and the partially purified EPS; and
c) removing the complex of step b) from the sample, thus removing the metal from the sample.

7. The method of claim 5, wherein the metal is uranium.

8. A method of chelating a metal in a sample, comprising the steps of:
a) contacting a metal containing sample with the partially purified EPS of claim 2; and
b) allowing a complex to form between the metal in the metal containing sample and the partially purified EPS, thus chelating a metal in the sample.

9. The method of claim 7, wherein the metal is uranium.

10. A method of producing the exopolysaccharide of claim 3 comprising the steps of:
a) culturing the Thauera MZ1T in medium; and
b) purifying the exopolysaccharide produced from the Thauera MZ1T culture of step a).

11. The method of claim 10, wherein the exopolysaccharide is purified by:
a) centrifuging the Thauera MZ1T culture;
b) decanting the supernatant;
c) concentrating the supernatant;
d) purifying the supernatant by size exclusion chromatography;
e) collecting an exopolysaccharide containing fraction produced from step (d), thus purifying the exopolysaccharide.

12. A method of removing a metal from a sample comprising:
a) contacting a metal containing sample with the exopolysaccharide of claim 3;
b) allowing a complex to form between the metal in the metal containing sample and the exopolysaccharide; and
c) removing the complex of step b) from the sample, thus removing the metal from the sample.

13. The method of claim 12, wherein the metal is uranium.

14. A method of chelating a metal in a sample, comprising the steps of:
a) contacting a metal containing sample with the exopolysaccharide of claim 3; and
b) allowing a complex to form between the metal in the metal containing sample and the exopolysaccharide, thus chelating a metal in the sample.

15. The method of claim 7, wherein the metal is uranium.
